(19) United States
(12) Patent Application Publication
(10) Pub. No.: US 2016/0139103 A1
(43) Pub. Date: May 19, 2016

(54) OIL IDENTIFICATION SYSTEM

(71) Applicant: Caterpillar Inc., Peoria, IL (US)

(72) Inventors: Amiyo K. Basu, Peoria, IL (US); Ian S. McInerney, Champaign, IL (US); Brian S. Howard, Williamsfield, IL (US); Ioan Paduret, Peoria, IL (US)

(73) Assignee: Caterpillar Inc., Peoria, IL (US)

(21) Appl. No.: 14/547,640

(22) Filed: Nov. 19, 2014

(51) Int. Cl. G01N 33/28; F01M 11/00; G01N 27/06; F01M 11/10

(52) U.S. Cl. CPC G01N 33/28; F01M 11/10; F01M 11/0004; G01N 27/06

(57) ABSTRACT

An oil identification system is disclosed. The oil identification system includes a parameter sensor coupled to at least one of an oil pan or an oil flow path of a machine. The oil identification system also includes an identification module. The identification module is also configured to receive a signal indicative of movement of charged particles in the oil of the at least one of the oil pan or the oil flow path. The identification module is further configured to determine a resistivity of the oil in the at least one of the oil pan or the oil flow path. The identification module is configured to compare the resistivity of the oil with resistivity data readings. The identification module is also configured to identify a type of oil in the at least one of the oil pan or the oil flow path based on the comparison.

### TECHNICAL FIELD

The present disclosure relates to an oil identification system, and more particularly to a system and method for identification of various types of oils used in engine applications.

### BACKGROUND

Oil is used for lubrication of machine parts, for example, an engine, a transmission system of the machine, a hydraulic system of the machine, and the like. The oil may be used to reduce wear of moving parts, reduce corrosion, improve sealing between mating parts, cleaning purposes, and cooling of machine parts.

In order to ensure satisfactory and reliable machine operation, manufacturers usually recommend the use of specific oil which is compatible with the machine parts. Sometimes, personnel may perform a fill operation of an incorrect or incompatible type of oil in the system. Use of oil that is incompatible with the machine parts may affect a performance of the machine and in some situations even lead to machine failure.

U.S. Pat. No. 5,656,767 describes a method and apparatus for automatically detecting a lubricant type and the relative quantity of water present in a test sample of lubricating oil includes the use of an open grid capacitive sensor element that incorporates the lubricating oil as a dielectric medium. The sensor element grid is energized by a frequency variable oscillator that automatically responds to changes in the oil dielectric constant with corresponding frequency changes. As a reference, a sample of new or uncontaminated test oil is confined in wet surface contact with the energized, open grid sensor element. Oscillator frequency changes are measured and recorded, either continuously or at frequent intervals, over a standardized elapsed time interval to generate a reference characterization of the frequency-time relationship distinctive of the particular oil. The same is repeated for a sample of contaminated oil and a corresponding frequency-time relationship generated. Water affinity rate correlations between frequency change and respective states of moisture content are determined and data base recorded. The affinity rate correlations are applied to the respective frequency differentials between the contaminated oil and uncontaminated oil at the end of the test period. Data bases are also recorded of correlations between natural frequency and sample temperature changes that distinguish lubricants by type, use classification or quantity of additive content.

### SUMMARY OF THE DISCLOSURE

In one aspect of the present disclosure, an oil identification system is disclosed. The oil identification system includes a parameter sensor coupled to at least one of an oil pan or an oil flow path of a machine. The parameter sensor is configured to generate a signal indicative of a measure of a movement of charged particles in oil of the at least one of the oil pan or the oil flow path. The oil identification system also includes an identification module communicably coupled to the parameter sensor. The identification module is configured to apply a voltage across the oil in the at least one of the oil pan or the oil flow path for generating the charged particles in the oil of the at least one of the oil pan or the oil flow path. The identification module is also configured to receive the signal indicative of the movement of the charged particles in the oil of the at least one of the oil pan or the oil flow path. The identification module is further configured to determine a resistivity of the oil in the at least one of the oil pan or the oil flow path based on the movement of the charged particles in the oil of the at least one of the oil pan or the oil flow path. The identification module is configured to compare the resistivity of the oil with resistivity data readings. The resistivity data readings comprise a number of resistivity readings for different types of oil, each of the resistivity readings measured at a set temperature. The identification module is also configured to identify a type of oil in the at least one of the oil pan or the oil flow path based on the comparison.

In one aspect of the present disclosure, a method of oil identification for at least one of an oil pan or an oil flow path of a machine is disclosed. The method includes applying a voltage across oil in the at least one of the oil pan or the oil flow path for generating charged particles in the oil of the at least one of the oil pan or the oil flow path. The method also includes receiving a signal indicative of a measure of a movement of the charged particles in the oil of the at least one of the oil pan or the oil flow path. The method further includes determining a resistivity of oil in the at least one of the oil pan or the oil flow path based on the movement of the charged particles in the oil of the at least one of the oil pan or the oil flow path. The method includes comparing the resistivity of the oil in the at least one of the oil pan or the oil flow path with resistivity data readings. The resistivity data readings comprise a number of resistivity readings for different types of oil, each of the resistivity readings measured at a set temperature. The method includes identifying a type of oil in the at least one of the oil pan or the oil flow path based, at least in part, on the comparison.

In one aspect of the present disclosure, an oil identification system for a machine is disclosed. The oil identification system includes at least one of an oil pan or an oil flow path. The oil identification system also includes a parameter sensor positioned in the at least one of the oil pan or the oil flow path. The parameter sensor is configured to generate a signal indicative of a measure of a movement of charged particles in oil of the at least one of the oil pan or the oil flow path. The oil identification system further includes an output unit. The oil identification system includes an identification module communicably coupled to the parameter sensor and the output unit. The identification module is configured to apply a voltage across the oil in the at least one of the oil pan or the oil flow path for generating the charged particles in the oil of the at least one of the oil pan or the oil flow path. The identification module is also configured to receive the signal indicative of the movement of the charged particles in the oil of the at least one of the oil pan or the oil flow path. The identification module is further configured to determine a resistivity of the oil in the at least one of the oil pan or the oil flow path based on the movement of the charged particles in the oil of the at least one of the oil pan or the oil flow path. The identification module is configured to compare the resistivity of the oil with resistivity data readings. The resistivity data reading comprises a number of resistivity readings for different types of oil, each of the resistivity readings measured at a set temperature. The identification module is also configured to identify a type of oil in the at least one of the oil pan or the oil flow path based on the comparison. The identification module is also configured to determine if the identified type of oil in the at least one of the oil pan or the oil flow path matches with a predetermined type of oil. The identification module is further configured to trigger an alert notification on the output unit, if the identified type of oil in the at least one of the oil pan or the oil flow path does not match the predetermined type of oil.

Other features and aspects of this disclosure will be apparent from the following description and the accompanying drawings.

### BRIEF DESCRIPTION OF THE DRAWINGS

FIG. 1 is a line diagram of an exemplary oil pan associated with an engine and a parameter sensor coupled to the oil pan, according to one embodiment of the present disclosure;

FIG. 2 is a line diagram of an exemplary oil flow path provided with a parameter sensor, according to one embodiment of the present disclosure;

FIG. 3 is a block diagram of an oil identification system, according to one embodiment of the present disclosure;

FIG. 4 is a graphical representation of an exemplary behavior of different types of oil based on temperature and resistivity readings, according to one embodiment of the present disclosure;

FIG. 5 is a graphical representation n of an exemplary behavior of an agglomeration quality of oil based on resistivity readings, according to one embodiment of the present disclosure; and

FIG. 6 is a flowchart for a method of oil identification, according to one embodiment of the present disclosure.

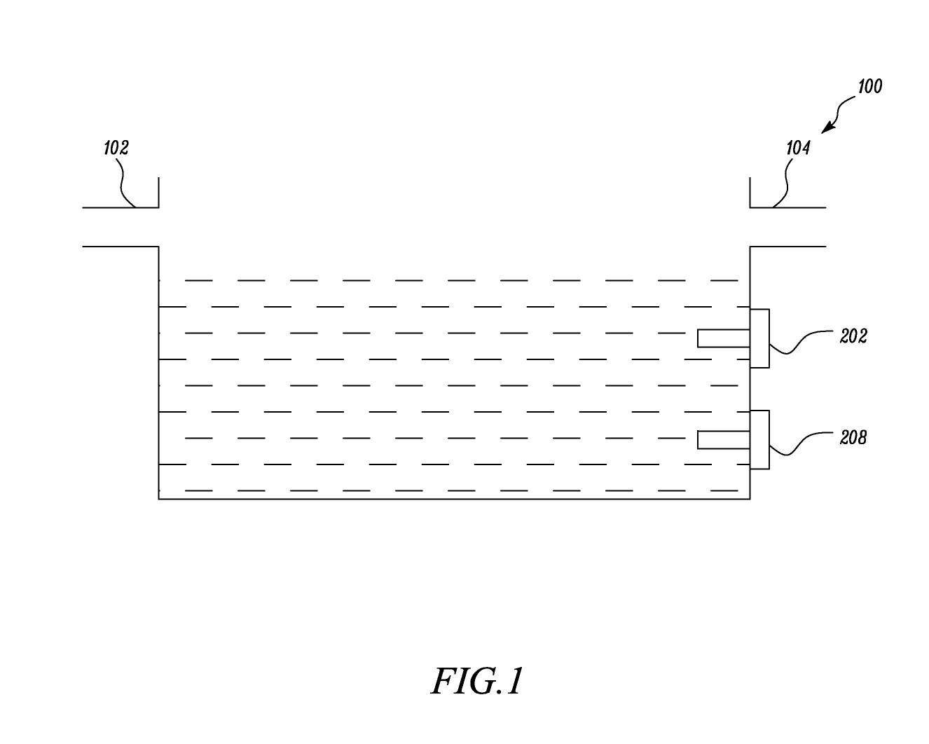

### DETAILED DESCRIPTION

Wherever possible, the same reference numbers will be used throughout the drawings to refer to the same or the like parts. FIG. 1 is a line diagram of an exemplary oil pan 100. The oil pan 100 is configured to hold oil therein. The oil from the oil pan 100 may be configured to flow through various parts of the engine system for lubrication, cooling or other purposes. In some examples, the oil from the oil pan 100 is used to lubricate various machine components or sub-systems of the machine, for example, but not limited to, an engine system, a transmission system, and a propulsion system. Alternatively, the oil pan 100 may be utilized in association with other equipment, such as, a generator, that may be driven by any power source to generate electricity. Based on the application, a type of oil stored in the oil pan 100 may vary without any limitation. For example, the type of oil may include a full synthetic oil, a blended synthetic oil, or a mineral oil, and so on.

The oil pan 100 has an inlet 102, such that the oil may be filled into the oil pan 100 therethrough. In one embodiment, the oil may be supplied into the oil pan 100 from an external source (not shown). The oil pan 100 includes an outlet 104 for discharging the oil from the oil pan 100 to various machine parts. It should be noted that the oil pan 100 depicted in the accompanying figures is exemplary. The structure, location, and functionality of the oil pan 100 may vary based on the application.

A parameter sensor 202 is coupled to the oil pan 100. In the illustrated embodiment, the parameter sensor 202 is mounted near a bottom of the oil pan 100. Alternatively, the parameter sensor 202 may be mounted at a different location from that shown in the accompanying figures. For example, the parameter sensor 202 may be mounted at the inlet 102 of the oil pan 100 for an early detection of oil type.

The parameter sensor 202 may be an electrochemical sensor. The parameter sensor 202 may be embodied as a probe having a pair of parallel plates or electrodes longitudinally spaced apart from each other. The plates may be made of a material that is compatible with the oil contacting the parameter sensor 202. When installed, the plates of the parameter sensor 202 are configured to contact the oil.

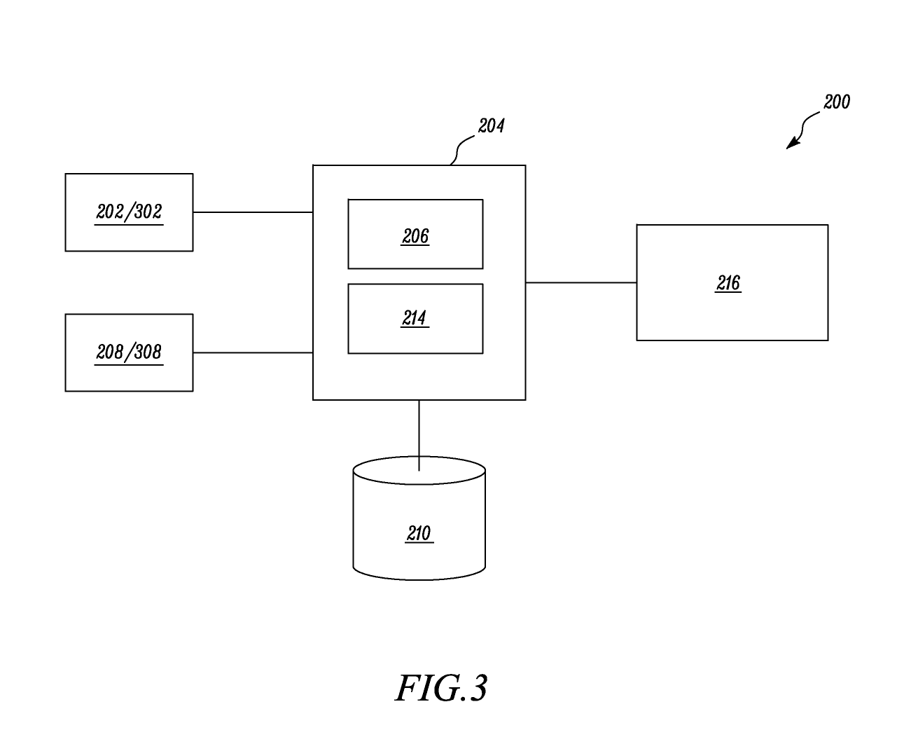

The present disclosure relates to an oil identification system 200 (see FIG. 3) for identifying the type of oil in the oil pan 100, based on signals received from the parameter sensor 202. The working of the oil identification system 200 will be explained later in this section connection with FIG. 3.

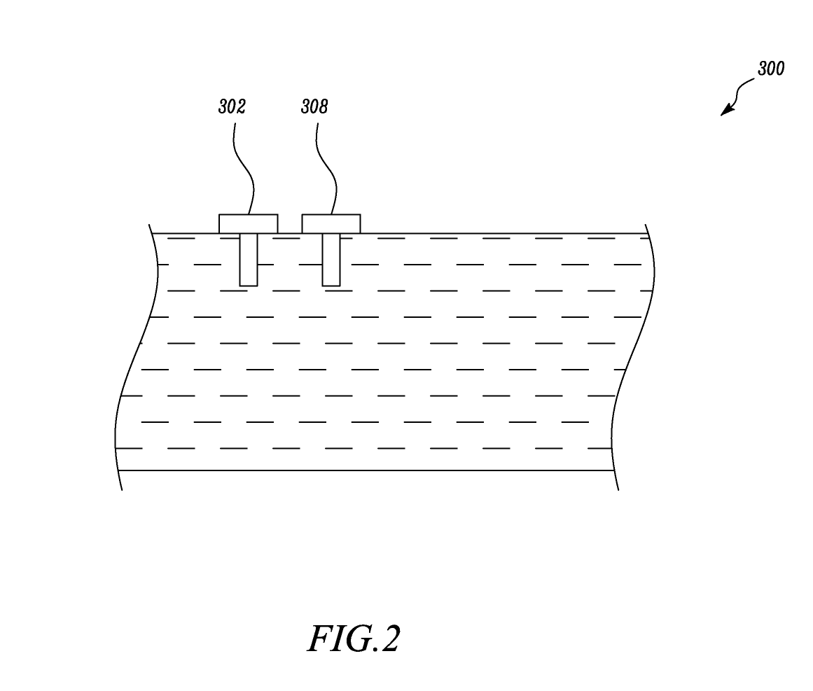

FIG. 2 is a line diagram of an exemplary oil flow path 300 associated with the engine of a machine. The oil flow path 300 may embody an oil passage that allows a flow of oil therethrough. In an exemplary embodiment, the oil flow path 300 may fluidly connect an oil storage equipment (not shown) associated with the machine to various machine portions.

As shown in FIG. 2, the parameter sensor 302 is mounted in the oil flow path 300, so that the oil flowing therethrough may contact the parameter sensor 302. In other embodiments, the parameter sensor 302 may be positioned within the oil flow path located before or after an oil filter (not shown). In yet another embodiment, the parameter sensor 302 may be mounted within the oil flow path positioned upstream or downstream of an oil cooler (not shown). The parameter sensor 302 of the oil flow path 300 may serve a similar functionality of the parameter sensor 202 explained above in connection with the oil pan 100.

The oil flow path 300 illustrated in FIG. 2 may serve as an alternate or additional environment in which the oil identification system 200 of the present disclosure may be employed in. Accordingly, the oil identification system 200 (see FIG. 3) may be utilized in a similar manner as in connection with the oil pan 100 described in relation with FIG. 1, to identify the type of oil in the oil flow path 300. It should be noted that the positioning of the parameter sensor 202, 302 within the oil pan 100 and the oil flow path 300 respectively that is shown in the accompanying figures is exemplary. The positioning of the parameter sensor 202, 302 may vary based on the type of application.

FIG. 3 illustrates a block diagram of an exemplary oil identification system 200 associated with the oil pan 100, the oil flow path 300, or both as the case may be. The oil identification system 200 is configured to identify the type of oil being filled into, stored within or flowing through the oil pan 100 and/or the oil flow path 300.

The oil identification system 200 includes an identification module 204. The identification module 204 is communicably coupled to the parameter sensor 202, 302 of the respective oil pan 100 or the oil flow path 300. The communication between the parameter sensor 202, 302 and the identification module 204 may be wired or wireless, based on the type of application. Further, the identification module 204 may be located on-board the machine or at a remote location.

The identification module 204 may embody any one of numerous commercially available microprocessors having detection, processing, and control capabilities. It should be appreciated that the identification module 204 may readily embody a general machine microprocessor capable of controlling numerous machine functions. A person of ordinary skill in the art will appreciate that the identification module 204 may additionally include other components and may also perform other functionality not described herein.

The identification module 204 is configured to apply a voltage across the oil in the oil pan 100 and/or the oil flow path 300 for generating charged particles in the oil of the oil pan 100 and/or the oil flow path 300. Due to the voltage applied, a movement of the charged particles is brought about in the oil of the oil pan 100 and/or the oil flow path 300. The parameter sensor 202, 302 is configured to generate a signal indicative of a measure of the movement of the charged particles in the oil of the oil pan 100 and/or the oil flow path 300.

Further, the identification module 204 may include a resistivity calculation unit 206. The resistivity calculation unit 206 is configured to receive the signal indicative of the movement of the charged particles in the oil of the oil pan 100 and/or the oil flow path 300 from the respective parameter sensor 202, 302. Based on the received signal, the identification module 204 is configured to determine a resistivity of the oil in the oil pan 100 and/or the oil flow path 300. The resistivity calculation unit 206 may be configured to calculate the resistivity of the oil. Based on the voltage applied and the movement of the charge particles in the oil, the resistivity calculation unit 206 may determine or calculate the resistivity in the oil of the oil pan 100 and/or the oil flow path 300 using methods known in the art. It should be noted that the oil identification system 200 of the present disclosure is configured to measure the resistivity of the oil based on thermal properties of the oil, more specifically based on the change in the electrochemical properties of the oil (that is, resistivity of the oil) with respect to temperature conditions and independent of time constraints.

Accordingly, the oil identification system 200 is configured to determine the resistivity of the oil in the oil pan 100 and/or the oil flow path 300 at a current temperature of the oil. Referring to FIGS. 1, 2 and 3, a temperature sensor 208, 308 may be coupled to the oil pan 100 and/or the oil flow path 300 respectively. In one example, the temperature sensor 208, 308 may be positioned such that the temperature sensor 208, 308 protrudes into the oil pan 100 and/or the oil flow path 300 respectively. Further, the temperature sensor 208, 308 may be positioned proximate to the respective parameter sensor 202, 302 in order to measure the temperature of the oil. The temperature sensor 208, 308 is configured to generate a temperature signal indicative of the current temperature of the oil in the oil pan 100 and/or the oil flow path 300 respectively. The temperature sensor 208, 308 may be communicably coupled to the identification module 204.

A database 210 may be communicably coupled to the identification module 204 in a wired or wireless manner. In one example, the database 210 may store resistivity data readings. The resistivity data readings may include resistivity readings corresponding to different types of oil taken at corresponding set temperatures. The location of the database 210 may vary based on the application. The resistivity data readings stored within the database 210 may be procured from any source and/or updated on a real time basis. The database 210 may be any conventional or non-conventional database known in the art. Moreover, the database 210 may be capable of storing and/or modifying pre-stored data as per operational and design needs. The data distribution may vary based on the application and the system may additionally include other databases that are not shown herein. The identification module 204 may retrieve the resistivity data readings stored in the database 210 to identify the type of oil.

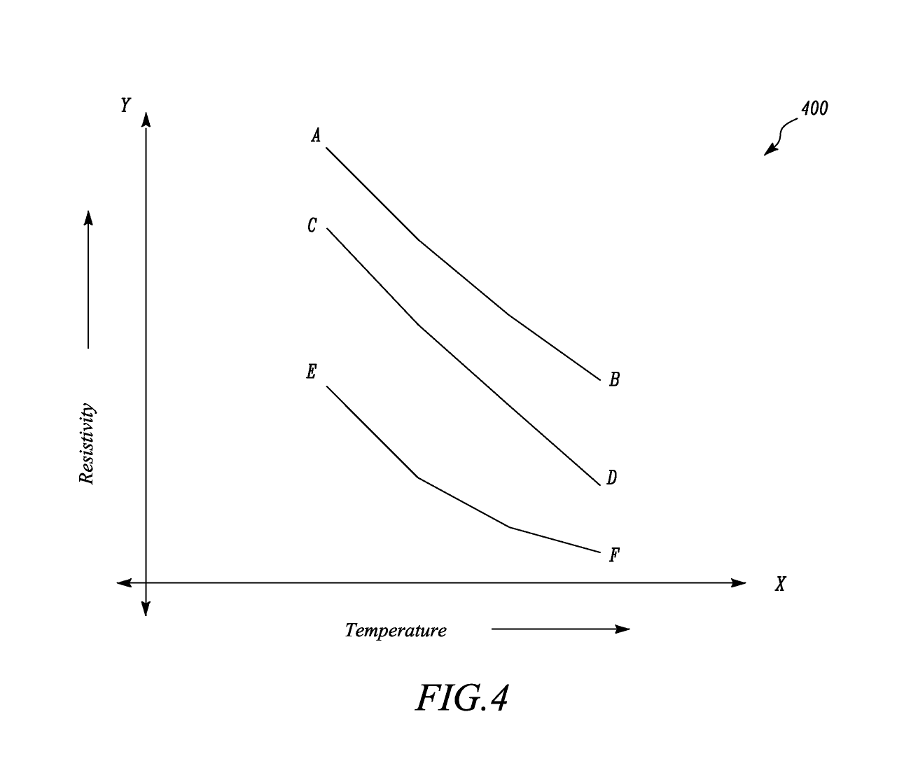

The identification module 204 includes a comparison unit 214. The comparison unit 214 is configured to receive the resistivity and temperature readings measured by the respective parameter sensor 202, 302 and the respective temperature sensor 208, 308. The comparison unit 214 may also retrieve the resistivity data readings from the database 210. Further, the comparison unit 214 compares the resistivity and temperature readings of the oil with the resistivity data readings, in order to identify the type of oil in the oil pan 100 and/or the oil flow path 300. The resistivity of different types of oil at the given current temperature is distinct from each other, and will be explained in detail in connection with FIG. 4. Accordingly, by determining the resistivity of the oil in the oil pan 100 and/or the oil flow path 300, the identification module 204 identifies the type of oil in the oil pan 100 and/or the oil flow path 300.

FIG. 4 illustrates an exemplary graphical relationship of the measured resistivity of different types of oil at corresponding temperature readings. The graph is a plot 400 of measured resistivity readings of oil (marked along Y axis) at different temperature readings of oil marked along (marked along X axis). As is illustrated in FIG. 4, curve AB, curve CD, and curve EF represent the behavioral change of resistivity of oil with change in temperature. More particularly, the curve AB represents the change in the resistivity of oil of synthetic type oil at different temperature readings. The curve CD represents the change in the resistivity of oil of blended type oil at different temperature readings. The curve EF represents the change in the resistivity of oil of mineral type oil at different temperature readings.

It should be noted that the curves AB, CD, EF as illustrated in the accompanying figures are exemplary in nature. The curves representing the given oil types may vary within a set threshold of the given curve. The graph in the accompanying drawings is exemplary in nature and does not limit the scope of the present disclosure. The plot 400 may additionally or alternatively be extended to include a group of curves within the set threshold of the illustrated curves for the given oil types. Further, based on the application, the resistivity data readings may include data for identification of still other types of oil, such as, near blended oil, near mineral oil, and so on. Alternatively or additionally, the resistivity data readings may also be utilized to detect if the oil is of degraded quality, which will be explained in detail later in this section.

Referring to FIG. 4, at a given temperature, the resistivity of different types of oil may be distinctly different from each other. Accordingly, based on the determination of the resistivity of the oil by the identification module 204; the identification module 204 may further determine the type of the oil by comparison with the resistivity data readings. The data or resistivity data readings may be stored in the database 210 for retrieval and identification of the type of oil in the oil pan 100 and/or the oil flow path 300.

An output unit 216 is communicably coupled to the identification module 204 in a wired or wireless manner. The output unit 216 is configured to receive information of the identified oil type from the identification module 204. The output unit 216 is also configured to provide an indication to a user, of the identified type of the oil. The output unit 216 may embody a visual output or an audio output. In one example, wherein the output unit 216 is embodied as a visual output, the output unit 216 may include a digital display device, an LCD device, an LED device, a CRT monitor, a touchscreen device or any other display device known in the art.

The output unit 216 may be mounted at a location such that the output unit 216 may be viewable to the user. In one embodiment, the output unit 216 may be a display device present in an operator cabin of the machine. Alternatively, the output unit 216 may be positioned on a frame of the machine, proximate to a location at which the oil may be introduced into the machine from the external source. The output unit 216 may notify the user regarding the identified oil type through a text message. In a situation wherein the output unit 216 is embodied as the audio output, an audio clip may be heard, thereby alerting the user of the identified oil type. It should be noted that the output unit 216 may include any other means other than those listed above.

The database 210 may be configured to store information of a predetermined type of oil which is compatible with the machine or system. In one embodiment, the comparison unit 214 is configured to retrieve the information of the predetermined type of oil from the database 210. The comparison unit 214 may compare and determine if the identified type of oil in the oil pan 100 and/or the oil flow path 300 matches with the predetermined type of oil. In a situation wherein the identified type of oil does not match with the predetermined type of oil, the identification module 204 triggers an alert notification to the user to inform the user of a discrepancy in the oil type. The alert notification may be provided via the output unit 216 explained earlier in this section.

A person of ordinary skill in the art will appreciate that the oil identification system 200 may be utilized in various applications, such as, for identification of different types of transmission oils, hydraulic oils, and so on. Further, other electrochemical properties of the oil may also be measured without deviating from the scope of the present disclosure.

The oil in the oil pan 100 and/or the oil flow path 300 may include a number of particles in the form of additives, such as, for example, detergents, dispersions, anti-wear agents, and the like. Properties of one or more of these additives may change during a course of oil usage. In some embodiments of the present disclosure, the oil identification system 200 is configured to determine the quality of the oil in the oil pan 100 and/or the oil flow path 300 based on the degradation of the particles in the oil.

The identification module 204 is configured to determine an agglomeration quality of the particles in the oil of the oil pan 100 and/or the oil flow path 300. In one example, the agglomeration quality of the particles may be determined by the resistivity of the oil which is determined by the resistivity calculation unit 206. Further, the database 210 may store a quality threshold that defines an acceptable agglomeration value of the oil. The identification module 204 may retrieve the quality threshold corresponding to the identified type of oil and compare the determined agglomeration quality with the quality threshold. Based on the comparison, the identification module 204 is configured to determine the quality of oil in the oil pan 100 and/or the oil flow path 300.

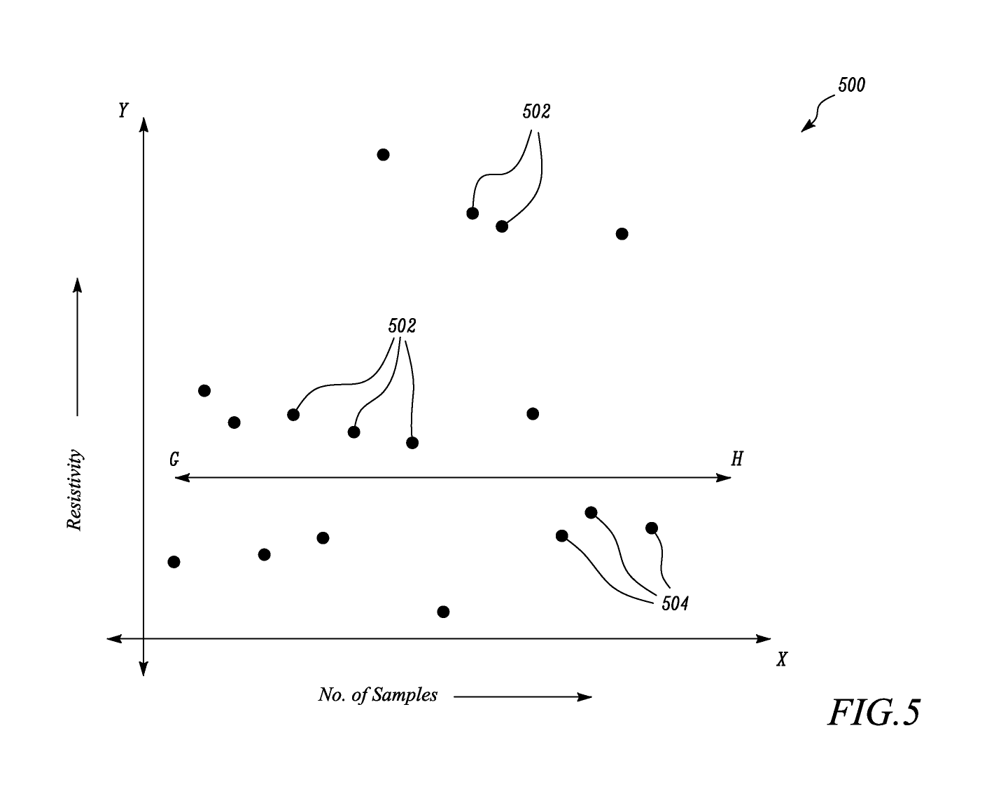

FIG. 5 illustrates an exemplary graphical relationship of the agglomeration quality of oil based on the resistivity of oil. The graph is a plot 500 of measured resistivity readings of oil (marked along Y axis) against number of and different samples of oil (marked along X axis). The plot 500 has a first set of agglomeration data-points 502 and a second set of agglomeration data-points 504 of the oil which lie above and below of the quality threshold GH respectively.

In a situation wherein the agglomeration data-points of oil lie above the quality threshold GH, the oil is determined to be of non-degraded quality. Accordingly, the oil samples having resistivity readings corresponding to the first set of agglomeration data-points 502 may be considered to have non-degraded quality. Further, the oil samples having resistivity readings corresponding to the second set of agglomeration data-points 504 may be considered to be of degraded quality, wherein the resistivity of such oil samples lies below the quality threshold GH. A person of ordinary skill in the art will appreciate that the quality of oil is based on the resistivity of oil and is independent of the type of oil. The graph in the accompanying figures is exemplary in nature and does not limit the scope of the present disclosure.

The identification module 204 may trigger an alert notification based on the determined quality of oil. For example, in a situation wherein the determined agglomeration quality of the oil in the oil pan 100 and/or the oil flow path 300 is lesser than the quality threshold GH, the identification module 204 may determine that the oil is of degraded quality. Accordingly, the identification module 204 may flag the alert, thereby notifying the user to take necessary actions. The alert notification may be provided to the user via the output unit 216.

### INDUSTRIAL APPLICABILITY

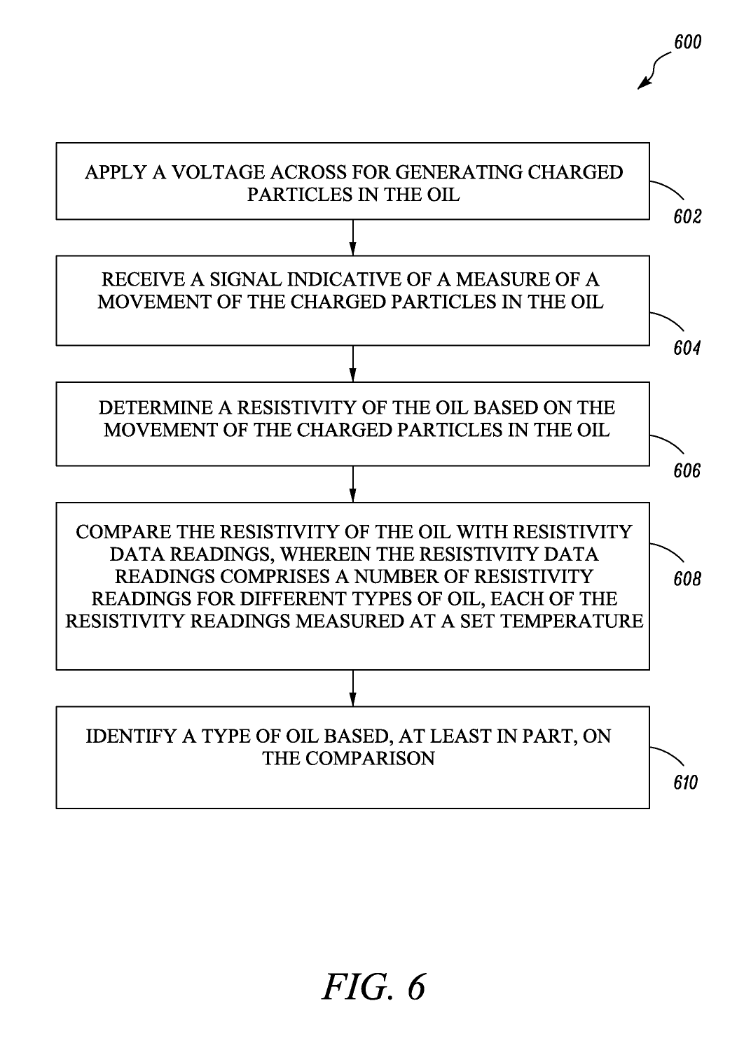

The present disclosure relates to the system and method for identification of the oil type based on the determined resistivity of the oil. FIG. 6 illustrates a method 600 of oil identification for the oil pan 100 and/or the oil flow path 300 of the machine. At step 602, the identification module 204 is configured to apply a voltage across the oil in the oil pan 100 and/or the oil flow path 300 for generating the charged particles in the oil of the oil pan 100 and/or the oil flow path 300. At step 604, the identification module 204 receives the signal indicative of the measure of the movement of the charged particles in the oil of the oil pan 100 and/or the oil flow path 300 from the parameter sensor 202, 302.

The type of oil in the oil pan 100 and/or the oil flow path 300 may include at least one of a full synthetic oil, a blended synthetic oil, or a mineral oil. Further, the identification module 204 also receives the temperature signal indicative of the current temperature of the oil in the oil pan 100 and/or the oil flow path 300 from the temperature sensor 208, 308.

At step 606, the identification module 204 is configured to determine the resistivity of the oil in the oil pan 100 and/or the oil flow path 300 based on the received signal. Also, the identification module 204 is configured to determine the resistivity of the oil in the oil pan 100 and/or the oil flow path 300 at the current temperature detected by the temperature sensor 208, 308. At step 608, the identification module 204 is configured to compare the resistivity of the oil in the oil pan 100 and/or the oil flow path 300 with the resistivity data readings. The resistivity data readings include a number of resistivity readings for different types of oil, wherein each of the resistivity readings is measured at a set temperature.

At step 610, based on the comparison, the identification module 204 is configured to identify the type of oil in the oil pan 100 and/or the oil flow path 300. The identification module 204 is also configured to determine if the identified type of oil in the oil pan 100 and/or the oil flow path 300 matches with the predetermined type of oil. In a situation wherein the identified type of oil in the oil pan 100 and/or the oil flow path 300 does not match the predetermined type of oil, the identification module 204 is configured to trigger the alert notification. The alert notification is provided to the user through the output unit 216, so that the user may take corrective actions.

The identification module 204 determines the agglomeration quality of the particles in the oil of the oil pan 100 and/or the oil flow path 300. Further, the identification module 204 is configured to compare the determined quality with the quality threshold. Based on the comparison, the identification module 204 is configured to determine the quality of oil in the oil pan 100 and/or the oil flow path 300, and trigger the alert notification.

The parameter sensor 202, 302 and the temperature sensor 208, 308 may be mounted at the entry point of the oil pan 100 and/or the oil flow path 300 to facilitate early detection. The oil identification system 200 may provide improved and accurate detection of the oil type thereby allowing the user or personnel to take timely measures in case of incorrect fill operations, thereby reducing or eliminating a risk of damage to the machine.

Further, a continual use of the degraded oil for machine applications may affect the performance of the parts of the machine. It may therefore be desirable for a user to be notified regarding the quality of the oil. The present disclosure also describes means for detection the quality of the oil in the oil pan 100 and/or the oil flow path 300. Accordingly, a probability of using degraded or deteriorated quality oil for machine application may be reduced, thereby increasing life expectancy of the machine components.

It should be noted that the present disclosure is not limited to the detection of oil type or the degradation of oil contained within the oil pan 100 and flowing in/out of the oil flow path 300, but may find various other applications not described herein. For example, the oil identification system 200 may be used for detection of oil type within a transfer case of a transmission system, or a storage device associated with a hydraulic system.

While aspects of the present disclosure have been particularly shown and described with reference to the embodiments above, it will be understood by those skilled in the art that various additional embodiments may be contemplated by the modification of the disclosed machines, systems and methods without departing from the spirit and scope of what is disclosed. Such embodiments should be understood to fall within the scope of the present disclosure as determined based upon the claims and any equivalents thereof.

What is claimed is:

1. An oil identification system comprising:
a parameter sensor coupled to at least one of an oil pan or an oil flow path of a machine, the parameter sensor configured to generate a signal indicative of a measure of a movement of charged particles in oil of the at least one of the oil pan or the oil flow path; and
an identification module communicably coupled to the parameter sensor, the identification module configured to:
apply a voltage across the oil in the at least one of the oil pan or the oil flow path for generating the charged particles in the oil of the at least one of the oil pan or the oil flow path;
receive the signal indicative of the movement of the charged particles in the oil of the at least one of the oil pan or the oil flow path;
determine a resistivity of the oil in the at least one of the oil pan or the oil flow path based on the movement of the charged particles in the oil of the at least one of the oil pan or the oil flow path;
compare the resistivity of the oil with resistivity data readings, wherein the resistivity data readings comprises a number of resistivity readings for different types of oil, each of the resistivity readings measured at a set temperature; and
identify a type of oil in the at least one of the oil pan or the oil flow path based on the comparison.

2. The oil identification system of claim 1 further comprising a temperature sensor configured to generate a temperature signal indicative of a current temperature of the oil in the at least one of the oil pan or the oil flow path, the temperature sensor being coupled to the identification module.

3. The oil identification system of claim 2, wherein the identification module is further configured to determine the resistivity of the oil in the at least one of the oil pan or the oil flow path at the current temperature.

4. The oil identification system of claim 1, wherein the identification module is further configured to:
determine if the identified type of oil in the at least one of the oil pan or the oil flow path matches with a predetermined type of oil; and
trigger an alert notification if the identified type of oil in the at least one of the oil pan or the oil flow path does not match the predetermined type of oil.

5. The oil identification system of claim 4 further comprising an output unit coupled to the identification module, the output unit configured to provide the alert notification to an operator.

6. The oil identification system of claim 1, wherein the type of oil in the at least one of the oil pan or the oil flow path includes at least one of a full synthetic oil, a blended synthetic oil, or a mineral oil.

7. The oil identification system of claim 1, wherein the identification module is further configured to:
determine an agglomeration quality of the oil of the at least one of the oil pan or the oil flow path based on the resistivity of the oil of the at least one of the oil pan or the oil flow path;
compare the determined agglomeration quality with a quality threshold; and
determine a quality of oil in the at least one of the oil pan or the oil flow path, based on the comparison.

8. The oil identification system of claim 7, wherein the identification module is further configured to trigger an alert notification based on the determined quality of oil.

9. The oil identification system of claim 7, wherein the quality of oil is determined as degraded oil if the agglomeration quality of the oil is less than the quality threshold.

10. A method of oil identification for at least one of an oil pan or an oil flow path of a machine, the method comprising:
applying a voltage across oil in the at least one of the oil pan or the oil flow path for generating charged particles in the oil of the at least one of the oil pan or the oil flow path;
receiving a signal indicative of a measure of a movement of the charged particles in the oil of the at least one of the oil pan or the oil flow path;
determining a resistivity of the oil in the at least one of the oil pan or the oil flow path based on the movement of the charged particles in the oil of the at least one of the oil pan or the oil flow path;
comparing the resistivity of the oil in the at least one of the oil pan or the oil flow path with resistivity data readings, wherein the resistivity data readings comprises a number of resistivity readings for different types of oil, each of the resistivity readings measured at a set temperature; and
identifying a type of oil in the at least one of the oil pan or the oil flow path based, at least in part, on the comparison.

11. The method of claim 10 further comprising:
receiving a temperature signal indicative of a current temperature of the oil in the at least one of the oil pan or the oil flow path.

12. The method of claim 11, wherein determining the resistivity step further comprises:
determining the resistivity of oil in the at least one of the oil pan or the oil flow path at the current temperature.

13. The method of claim 10 further comprising:
determining if the identified type of oil in the at least one of the oil pan or the oil flow path matches with a predetermined type of oil; and
triggering an alert notification if the identified type of oil in the at least one of the oil pan or the oil flow path does not match the predetermined type of oil.

14. The method of claim 13 further comprising:
providing the alert notification to an operator through an output unit.

15. The method of claim 10, wherein the type of oil in at least one of the oil pan or the oil flow path includes at least one of a full synthetic oil, a blended synthetic oil, or a mineral oil.

16. The method of claim 10 further comprising:
determining an agglomeration quality of particles in the oil of the at least one of the oil pan or the oil flow path based on the resistivity of the oil of the at least one of the oil pan or the oil flow path;
comparing the determined agglomeration quality with a quality threshold; and
determining a quality of oil in the at least one of the oil pan or the oil flow path, based on the comparison.

17. The method of claim 16 further comprising triggering an alert notification based on the determined quality of oil.

18. An oil identification system for a machine, the oil identification system comprising:
a parameter sensor positioned in at least one of an oil pan or an oil flow path, the parameter sensor configured to generate a signal indicative of a measure of a movement of charged particles in oil of the at least one of the oil pan or the oil flow path;
an output unit; and
an identification module communicably coupled to the parameter sensor and the output unit, the identification module configured to:
apply a voltage across the oil in the at least one of the oil pan or the oil flow path for generating the charged particles in the oil of the at least one of the oil pan or the oil flow path;
receive signal indicative of the movement of the charged particles in the oil of the at least one of the oil pan or the oil flow path;
determine a resistivity of the oil in the at least one of the oil pan or the oil flow path based on the movement of the charged particles in the oil of the at least one of the oil pan or the oil flow path;
compare the resistivity of the oil in the at least one of the oil pan or the oil flow path with resistivity data readings, wherein the resistivity data readings comprises a number of resistivity readings for different types of oil, each of the resistivity readings measured at a set temperature;
identify a type the oil in the at least one of the oil pan or the oil flow path based on the comparison;
determine if the identified type of oil in the at least one of the oil pan or the oil flow path matches with a predetermined type of oil; and
trigger an alert notification on the output unit, if the identified type of oil in the at least one of the oil pan or the oil flow path does not match the predetermined type of oil.

19. The oil identification system of claim 18, wherein the type of oil includes at least one of a full synthetic oil, a blended synthetic oil, or a mineral oil.

20. The oil identification system of claim 18, wherein the identification module is further configured to:
determine an agglomeration quality of particles in the oil of the at least one of the oil pan or the oil flow path based on the resistivity of the oil of the at least one of the oil pan or the oil flow path;
compare the determined agglomeration quality with a quality threshold; and
determine a quality of oil in the at least one of the oil pan or the oil flow path, based on the comparison.
